(19) United States
(12) Patent Application Publication
(10) Pub. No.: US 2002/0147496 A1
(43) Pub. Date: Oct. 10, 2002

(54) Apparatus for treating spinal discs

(75) Inventors: W. Martin Belef, San Jose, CA (US); Stephen M. Salmon, Napa, CA (US); William N. Aldrich, Napa, CA (US); Richard S. Ginn, San Jose, CA (US); Ronald J. Jabba, Redwood City, CA (US)

Correspondence Address: LYON & LYON LLP, 633 WEST FIFTH STREET, SUITE 4700, LOS ANGELES, CA, 90071, US

(73) Assignee: Integrated Vascular Systems, Inc.

(21) Appl. No.: 09/827,427

(22) Filed: Apr. 6, 2001

(51) Int. Cl. A61F 2/44

(52) U.S. Cl. 623/17.12

(57) ABSTRACT

Apparatus and methods for treating a spinal disc are disclosed. An opening is created in the annulus fibrosis, and nucleus pulposus is removed from the interior of the disc. The interior is lined with a nonporous, bioabsorbable liner, and filled with a fill material, such as nucleus pulposus, to cause the liner to expand to engage tissue surrounding the interior. The liner may be a sheet of extra-cellular matrix material that is introduced into-the interior, or a bladder of extra-cellular matrix material including a neck communicating with an interior region of the bladder. The sheet or bladder may be carried by a delivery device, e.g., a catheter or rod. After the interior region is filled, the opening is closed using a plug or other closure device. The plug may include threads on its external surface for securing the plug in the opening.

### FIELD OF THE INVENTION

The present invention relates generally to treatment of spinal discs, and more particularly to apparatus and methods for treating ruptured or degenerated spinal discs.

### BACKGROUND

Various apparatus and methods have been suggested for treating spinal discs when they degenerate or otherwise become injured. For example, spinal fixation, i.e., fixing the vertebrae on either side of an injured disc relative to one another, is a commonly used treatment. This may involve inserting pedicle screws or other anchors into the vertebrae, and securing rods, wires, cages, and the like between the vertebrae, thereby substantially removing much of the forces acting on the disc during subsequent activity by the patient. Such fixation procedures, however, may substantially impair free movement by the patient, because relative movement of the vertebrae is intentionally fixed.

As an alternative to fixation, an injured disc may be completely removed and replaced with a prosthesis. Exemplary prosthetic discs and methods for implanting them are disclosed in U.S. Pat. Nos. 4,863,477, issued to Monson, 5,123,926, issued to Pisharodi, and 6,146,419, issued to Eaton.

U.S. Pat. Nos. 5,549,679 and 5,571,189, issued to Kuslich, disclose implanting a porous bag into a spinal disc to promote fusion of the adjacent vertebrae. A bore is formed through the annulus fibrosis to gain access to the interior of the annulus. A hollow space is formed within the interior of the annulus that exposes surface areas of the vertebrae on either side of the to disc. A porous bag is inserted into the space and filled with finely chopped cancelous bone chips. The bag is formed from a porous fabric or a polymeric material having a plurality of perforations formed therein to promote bone ingrowth into the space and ensure that fusion occurs.

Once the bag is filled to a desired pressure, the inlet to the bag is sealed using a threaded cap, a purse-string closure, a staple, or tying a knot in the bag. A patch is then attached to the exterior of the annulus fibrosis in an attempt to seal the entry passage used to access the interior of the disc. Because of the significant stresses experienced by spinal discs during normal physical activity, however, such patches may not resist the substantial pressure experienced within a spinal disc during normal physical activity.

Thus, similar to fixation, Kuslich merely proposes fusing the adjacent vertebrae on either side of the disc being treated. As with conventional fixation, fusion may substantially impair free movement by the patient after the treated site has healed, and does not restore the spinal disc to an otherwise healthy state that may support normal movement.

U.S. Pat. No. 6,022,376, issued to Assell et al., discloses implanting a capsule-shaped prosthetic implant within a spinal disc. The implant is formed from a polymer jacket containing a polymer core, such as hydrogel, that is in a flowable state. Similar to Kuslich, the jacket may be inserted into a space within a spinal disc, and then polymer core may be introduced into the jacket after implantation within the disc. Alternatively, the jacket, already filled with the polymer core, may be implanted within the disc space. The result is a substantially permanent implant that is intended to act as a spacer and cushion.

U.S. Pat. No. 5,964,807, issued to Gan et al. discloses implanting “hybrid” material directly within a space created within a spinal disc. The hybrid material includes bioactive glass granules that are intended to promote cell growth and enhance growth of bone cells. The bioactive glass granules may be mixed with other materials, such as invertebral disc cells, such as nucleus pulposus material, growth factors to promote cell growth, and/or polymer materials. Similar to Kuslich, however, the intended result is fusion of the adjacent vertebrae and not restoration of the spinal disc to normal health.

U.S. Pat. Nos. 4,772,287 and 4,904,260, issued to Ray et al., disclose a pair of capsules that may be implanted within a spinal disc. Each capsule has a bladder that may be filled with a fluid including a therapeutic agent. The bladder has a semipermeable membrane that has a pore size that blocks flow of human cells but permits passage of therapeutic agents slowly through the membrane.

Accordingly, apparatus and methods for treating spinal discs would be considered useful.

### SUMMARY OF THE INVENTION

The present invention is directed to apparatus and methods for treating spinal discs. In accordance with one aspect, an apparatus is provided that includes an inflatable bladder including a neck defining an opening communicating with an interior of the bladder. A sealing member may be provided for securing the neck over the distal end of the tubular member and/or for sealing the neck after the bladder is filled. For example, the sealing member may be an elastic ring biased to constrict the neck upon withdrawal of the distal end of the tubular member from within the neck. Preferably, the neck is substantially everted within the interior of the bladder, and the elastic ring is disposed around the everted neck within the interior of the bladder.

In a preferred embodiment, the bladder is formed from bioabsorbable material, e.g., intestinal submucosa, stomach submucosa and bladder submucosa. The bladder may also be substantially inelastic material and/or may be substantially nonporous. The bladder may be expandable from a collapsed configuration to facilitate introduction into a spinal disc to an enlarged configuration for filling a cavity created within the spinal disc. Preferably, the bladder generally assumes a disc shape including convex opposing surfaces in the enlarged configuration.

The apparatus may also include a delivery device for delivering the bladder into a spinal disc. The delivery device generally includes a tubular member including a proximal end, a distal end having a size for insertion through an opening into a spinal disc, and a lumen extending between the proximal and distal ends. The neck of the bladder is detachably connected to the distal end of the tubular member such that the interior of the bladder communicates with the lumen. A source of fill material may be provided, e.g., connected to the proximal end of the tubular member and communicating with the lumen. In a preferred embodiment, the fill material includes nucleus pulposus, preferably including at least some of the nucleus pulposus material removed from the spinal disc being treated. In addition, or alternatively, the fill material may include other materials, such as autologous therapeutic agents, e.g., concentrated growth factors, extra-cellular matrix material, e.g., intestinal submucosa, stomach submucosa and bladder submucosa, saline, a pharmaceutical, genetic material, and the like.

The delivery device may also include a sheath slidably disposed over the tubular member. The sheath may include a distal region for receiving the bladder therein in a collapsed configuration. The delivery device may also include a pusher member slidable along the tubular member, the pusher member configured for directing the neck off of the distal end of the tubular member. For example, the pusher member may include a substantially blunt distal end for engaging the neck when the distal end of the tubular member is withdrawn from within the neck.

In an alternative embodiment, the distal end of the tubular member may include one or more electrodes for delivering energy to tissue surrounding a passage through which the tubular member is inserted for closing the passage upon withdrawal of the tubular member. In this embodiment, the apparatus may also include a source of energy, e.g., a radio frequency (RF) generator, coupled to the electrodes for providing the energy. The distal end of the tubular member may also include a radiopaque marker.

In accordance with another aspect of the present invention, a method is provided for treating a spinal disc of a patient, e.g., using an apparatus such as that described above. Generally, the spinal disc includes an annulus fibrosis and nucleus pulposus within an interior region defined by the annulus fibrosis. First, the spinal disc to be treated is accessed, and an opening is created in the annulus fibrosis to access the interior region of the annulus fibrosis. At least a portion of, and preferably substantially all of, the nucleus pulposus material is removed from the interior region of the annulus fibrosis to define a space.

The space is lined with a substantially nonporous, bioabsorbable liner material, and filled with a fill material sufficient to cause the liner material to expand to substantially engage tissue surrounding the space. For example, the liner material may be a sheet of substantially nonporous, bioabsorbable material, such as an extra-cellular matrix. Alternatively, a substantially nonporous, bioabsorbable bladder, such as that described above, may be introduced within the space in a collapsed configuration, e.g., within a delivery device. The bladder may be filled with a fill material sufficient to cause the bladder to expand to an enlarged configuration to substantially occupy the space and/or engage surrounding tissue as it is filled.

Preferably, the fill material includes nucleus pulposus, e.g., nucleus pulposus removed from the disc. In addition, the fill material may also include naturally occurring extra-cellular matrix material, such as intestinal submucosa, stomach submucosa and bladder submucosa, and/or other materials, such as saline, a pharmaceutical, autologous therapeutic agents, genetic material, and/or other materials, e.g., to promote healing. Alternatively, the fill material may be a polymer, such as interpenetrating polymer network (IPN) material.

In a further alternative, before the liner material is introduced into the interior region, a flowable fill material may be introduced into the interior region of the disc. Preferably, the fill material includes naturally occurring extra-cellular matrix material, such as intestinal submucosa, stomach submucosa and bladder submucosa. The flowable fill material may be a slurry also including saline and/or other materials to promote healing. As the liner material or bladder is filled, it may force the fill material within the interior region to fill any gaps or fissures, e.g., in the annulus fibrosis.

After the space within the disc has been filled with fill material, the opening may be closed. This may involve applying energy, e.g., RF energy, to the annular fibrosis tissue surrounding the opening. Alternatively, it may involve deploying a closure element to close the opening.

In a further alternative, a tubular plug member may be provided on the bladder, e.g., bonded or otherwise attached to the neck of the bladder. In one embodiment, the plug member may include a lumen communicating with an interior region of the bladder. The plug member may include a thread pattern on its external surface for substantially securing the plug member into the opening, e.g., by threading the plug member into tissue surrounding the opening. A cannula or other tubular member may be inserted into the lumen for facilitating introduction of fill material into the bladder through the lumen.

In another embodiment, the lumen of the plug member may be closed, e.g., by deploying an internal plug element within the lumen of the plug member. For example, a ball may be stored in a pocket in the plug member communicating with the lumen, the ball being coupled to a filament extending from the lumen. The filament may be pulled to deploy the ball within the lumen to substantially seal the lumen from fluid flow therethrough.

In still another embodiment, the space within the disc may be lined by introducing a sheet of substantially nonporous, bioabsorbable material into the space such that an outer edge of the sheet extends through the opening. Any excess sheet material extending from the opening may be trimmed, e.g., before or after closing the opening. A plug may be introduced into the opening, e.g., to substantially engage the sheet against the surrounding tissue and/or to substantially close the opening. The plug may include a thread pattern, allowing the plug to be threaded into the opening, or other expandable elements that may engage surrounding tissue and/or otherwise substantially close the opening.

In an alternative embodiment, an elongate member may be used to both fill the interior of the disc and to close the opening providing access to the interior. The elongate member may include a plug member, such as one of those described elsewhere herein, and an elongate body of fill material attached to one end of the plug member. For example, the body of fill material may include one or more layers of naturally occurring extra-cellular matrix material and/or nucleus pulposus rolled or packed into a tubular or substantially solid body. The body of fill material is sufficiently flexible that it may be introduced through the opening and packed into the interior of the disc to substantially fill the interior, e.g., to a predetermined pressure.

Preferably, the body of fill material may be provided in a predetermined length or cut to a predetermined length having a volume substantially similar to a volume of the interior of the disc. The body of fill material may be introduced through the opening, until the plug member is received and/or secured in the opening to substantially close the opening. When the plug member is secured within the opening, the body of fill material preferably substantially fills the interior of the disc, the plug member preventing substantial leakage of the fill material from the interior.

In accordance with yet another aspect of the present invention, a method is provided for treating a spinal disc of a patient, e.g., using one or more therapeutic agents. A stylet including a pointed distal end is inserted through the annulus fibrosis to create a passage communicating with the interior region of the disc. A tubular member is advanced over the stylet until a distal end of the tubular member is disposed within the interior region. The stylet is withdrawn from within the tubular member, and a therapeutic agent is introduced through the tubular member into the interior region.

A single bolos of therapeutic agent may be delivered into the interior region, or a series of treatments may be provided. For example, a pump, which may be implanted within the patient's body, may be connected to the tubular member, and the therapeutic agent may be delivered by the pump into the interior region over a predetermined time.

Upon completion of delivery of the therapeutic agent, the tubular member may be withdrawn from the interior region, and the passage may be closed. The passage may be closed by applying energy to annular fibrosis tissue surrounding the passage to close the passage and/or by deploying a closure element, as described above.

In accordance with still another aspect of the present invention, an apparatus for closing a passage through tissue is provided. The apparatus includes an energy delivery device, a needle, and a syringe. The energy delivery device includes a handle member having a connector on its distal end, the connector including an electrically conductive region. An electrically insulated elongate element extends from the distal that terminates in an uninsulated distal tip.

During use, the needle is connected to the syringe, and then is inserted through tissue. A therapeutic agent is delivered through the needle, and then the syringe is disconnected from the needle. The elongate element is inserted into the needle until the distal tip extends beyond the distal end of the needle, and the connector connects the needle to the conductive region. Electrical energy is delivered from a source of electrical energy via the distal tip and the needle to tissue surrounding the passage to close the passage as the needle is withdrawn. The apparatus may be used to close openings, particularly needle tracks, preferably through annular fibrosis of a spinal disc into an interior of the disc. The apparatus may also be used to close openings through other tissues, for example, through cardiac tissues.

Other objects and features of the present invention will become apparent from consideration of the following description taken in conjunction with the accompanying drawings.

### BRIEF DESCRIPTION OF THE DRAWINGS

FIGS. 1A-1D are cross-sectional side views of a first preferred embodiment of an apparatus for treating a spinal disc, in accordance with the present invention.

FIGS. 1E and 1F are cross-sectional views of alternative embodiments of an inflated bladder for use with the apparatus of FIGS. 1A-1D.

FIGS. 2A-2I are cross-sectional side views of a spinal disc being treated using the apparatus of FIGS. 1A-1D.

FIG. 3A shows a preferred embodiment of an implant for treating a spinal disc, in accordance with the present invention.

FIGS. 3B-3D are cross-sectional side views of a spinal disc, showing a method for treating a spinal disc using the implant of FIG. 3A.

FIGS. 4A and 4B are side and cross-sectional views, respectively of another apparatus for treating a spinal disc, in accordance with the present invention.

FIG. 5 is a cross-sectional view of a spinal disc being treated with the apparatus of FIGS. 4A and 4B.

FIG. 6 is a side view of an implant for treating a spinal disc, in accordance with the present invention.

FIG. 7 is a cross-sectional view of a spinal disc being treating using the implant of FIG. 6.

FIGS. 8A-8C are cross-sectional top views of a spinal disc, showing a method for introducing therapeutic agents into the spinal disc, in accordance with the present invention.

FIG. 9 shows a kit, including a syringe, a needle, and an energy delivery device for treating a spinal disc, in accordance with the present invention.

FIGS. 10A-10C are cross-sectional views of a spinal disc being treated using the kit of FIG. 9.

### DETAILED DESCRIPTION OF THE PREFERRED EMBODIMENTS

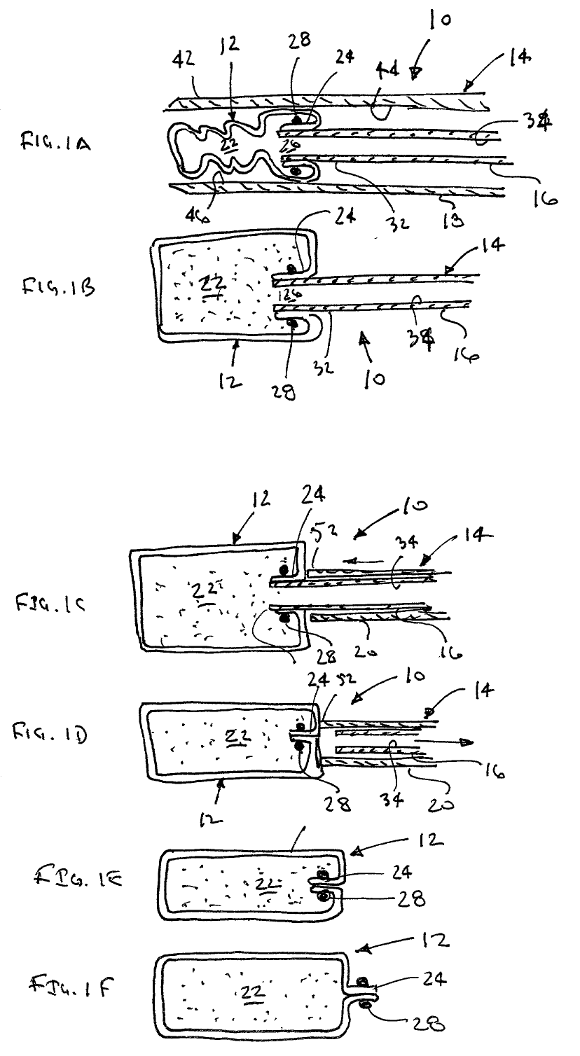

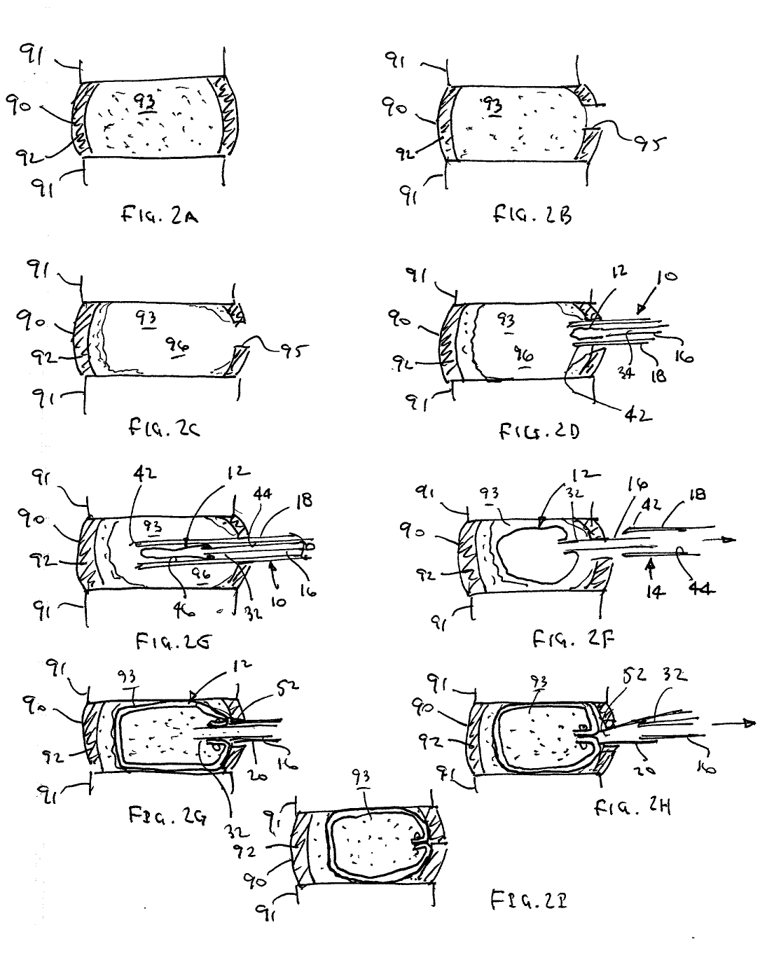

Turning now to the drawings, FIGS. 1A-1D show a first preferred embodiment of an apparatus 10 for treating a spinal disc (not shown), in accordance with the present invention. The apparatus 10 generally includes an inflatable bladder 12 and a delivery device 14, which may include a catheter 16, a delivery sheath 18, and/or a pusher member 20.

Generally, the bladder 12 is a substantially enclosed body defining an interior space 22. A neck 24 extends from the bladder 12 that defines an opening 26 communicating with the interior space 22. A sealing member 28 may be provided on the neck 24 for substantially sealing the opening 26. For example, an elastic ring may be provided around the neck 24 that is biased to constrict and thereby automatically close the opening 26. The elastic ring may be formed from a biocompatible material, such as a metal, e.g., stainless steel or Nitinol, or a polymer, and/or a bioabsorbable material, such as those described below. Alternatively, the sealing member 28 may be one or more filaments (not shown) attached or woven into the neck 24 that may be selectively tightened to close the opening 26. Adhesives or other sealants may also be provided, either alone or in conjunction with the sealing member 28.

In one embodiment, the neck 24 is everted within the interior space 22 of the bladder 12, and the sealing member 28 is disposed around the neck 24 within the interior space 22, as shown in FIG. 1E. Alternatively, the neck 24 may extend outwardly away from the bladder 12, as shown in FIG. 1F, and the sealing member 28 may be located around the neck 24 outside the bladder 12. In a further alternative, the neck may be eliminated, and an opening (not shown) may be provided directly in a wall of the bladder 12 to provide access into the interior space 22. In this embodiment, the opening may be sealed in a number of ways, e.g., by plugging the opening with a plug or other material, by pulling the wall around the opening closed, and stitching, bonding, or fusing the wall together, and the like (not shown).

The bladder 12 is generally expandable from a collapsed configuration, such as that shown in FIG. 1A, which may facilitate its introduction into a spinal disc, to an enlarged configuration, such as that shown in FIG. 1E. Preferably, the bladder 12 is formed from a substantially inelastic material that assumes a predetermined shape in the enlarged condition. For example, the bladder 12 may generally assume a circular disk shape that may correspond substantially to the shape of a spinal disc within which the bladder 12 is implanted. For example, the bladder 12, similar to natural intervertebral discs, may have a disc shape including convex upper and lower surfaces, e.g., having a greater thickness in its middle region than its outer edges. In a preferred embodiment, in the enlarged configuration, the bladder 12 has a diameter between about one and six centimeters (1-6 cm) and a height between about a half centimeter and three centimeters (0.5-3.0 cm).

Alternatively, the bladder 12 may be formed from an elastic material such that the bladder 12 may substantially fill a space within which it is inflated. In this embodiment, the bladder 12 may be inflated to one of a range of sizes, e.g., for filling a cavity having a variety of sizes and shapes.

The wall of the bladder 12 is preferably substantially nonporous, thereby preventing fluid passage therethrough and/or tissue-ingrowth. Alternatively, the wall of the bladder 12 may be porous to selected materials, such as proteoglycans, while being substantially nonporous to other materials. The bladder 12 may be formed from a biocompatible material, and preferably from a bioabsorbable material, such as intestinal submucosa, stomach submucosa, bladder submucosa, and/or other extra-cellular matrices (ECM's).

Returning to FIG. 1A, the catheter 16 of the delivery device 14 generally includes a substantially rigid or semi-rigid tubular member having a proximal end (not shown), a distal end 32 having a size for insertion through an opening into a spinal disc, and a lumen 34 extending between the proximal end and the distal end 32. The proximal end may include a handle or other mechanism (not shown) for manipulating the catheter 16. In addition, the proximal end may include a seal (not shown) for selectively closing the lumen 34 and/or a port for connecting to a source of fill material (not shown). The catheter 16 and/or its various components may be formed from a variety of known biocompatible materials, e.g., metals, such as stainless steel, and/or polymers or other plastics.

The bladder 12 is generally carried by the distal end 32 of the catheter 16, e.g., by inserting the distal end 32 into the neck 24. The sealing member 28 may substantially secure the neck 24 over the distal end 32 of the catheter 16 and/or substantially seal the opening 26. A source of fill material (not shown) may be connected to the proximal end, the source communicating with the lumen 34 for delivering fill material, e.g., including nucleus pulposus, to the distal end 32 of the catheter 16. Thus, with the neck 24 of the bladder 12 secured over the distal end 32 of the catheter 16, the fill material may be selectively introduced into the interior space 22 of the bladder 12 to fill and expand the bladder 12. The source of fill material may include a manual device, such as a syringe (not shown), a powered device, such as a pump (not shown), and the like.

The sheath 18 is a tubular member including a proximal end (not shown), a distal end 42 having a size for insertion into a spinal disc, and a lumen 44 extending between the proximal and the distal ends 42. The lumen 44 is sufficiently large such that the sheath 18 is slidable over the catheter 16, as shown in FIG. 1A. When the catheter 16 is fully received within the sheath 18, the lumen 44 preferably defines a distal region 46 beyond the distal end 32 of the catheter 16 for receiving the bladder 12 therein, also as shown in FIG. 1A.

As shown in FIGS. 1C and 1D, the pusher member 20 is a tubular member that is generally slidable over the catheter 16. Preferably, the pusher member 20 slidably engages an outer surface of the catheter 16 for facilitating release of the bladder 12 from off of the distal end 32. For example, the pusher member 20 may have a substantially blunt distal end 52 for abutting the neck 24 of the bladder 12 during withdrawal of the catheter 16, as described further below. Alternatively, other pusher members, e.g., including gripping elements, may be provided that may engage or be selectively secured to the neck 24 during withdrawal of the distal end 32 of the catheter 16.

In alternative embodiments, the catheter 16 may include one or more electrodes (not shown) on the distal end 32. For example, a single electrode (not shown) may be provided on the distal end 32, e.g., on the distal-most tip of the catheter 16. An external electrode may then be provided, e.g., a conductive pad in contact with the patient's skin (not shown), that may be electrically coupled to the electrode via the patient's tissue, e.g., for uni-polar operation. Alternatively, a plurality of electrodes (not shown) may be provided that are disposed axially a predetermined distance from one another on the distal end 32, e.g., for bi-polar operation.

The electrode(s) may be used for delivering energy to tissue surrounding a passage through which the catheter 16 is inserted, e.g., for closing the passage upon withdrawal of the catheter 16 and/or for closing the opening 26 in the bladder 12, as described further below. A source of energy, such as a radio frequency (RF) generator, may be coupled to the electrode(s), e.g., via a wire or other conductor extending within a lumen (not shown) or wall of the catheter 16, e.g., between the proximal and distal ends 32.

A temperature sensor, such as a thermocouple or thermistor (not shown), may also be provided on the distal end 32 of the catheter 16, e.g., for monitoring delivery of energy via the electrode(s). In addition or alternatively, a marker, such as a radiopaque band, may be provided at a predetermined location on the distal end 32 of the catheter 16, e.g., for monitoring the position of the electrode(s) before applying energy to close the passage.

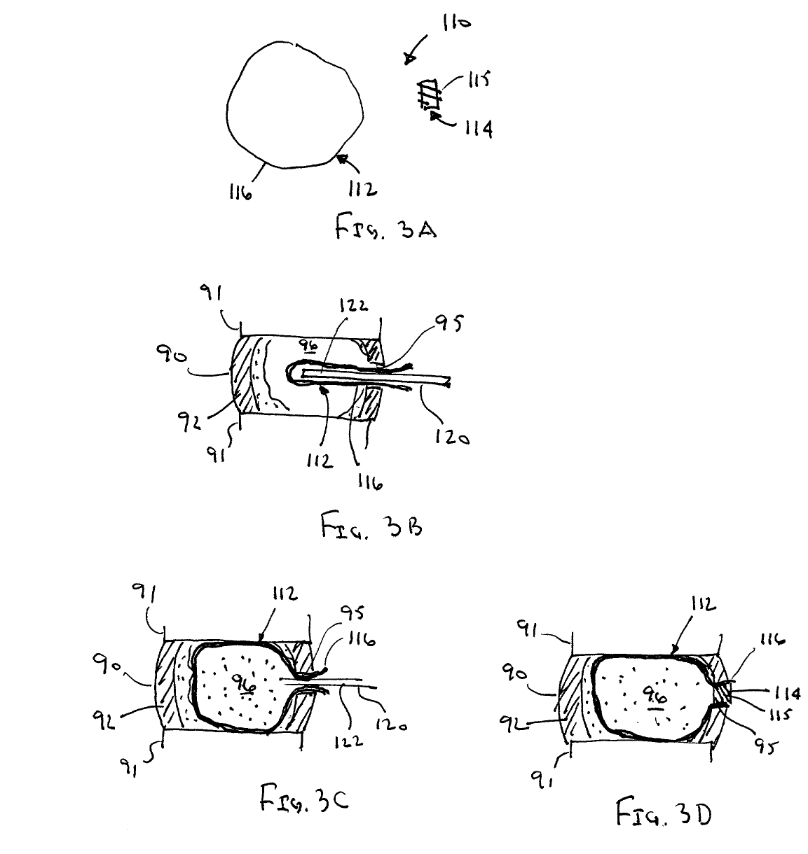

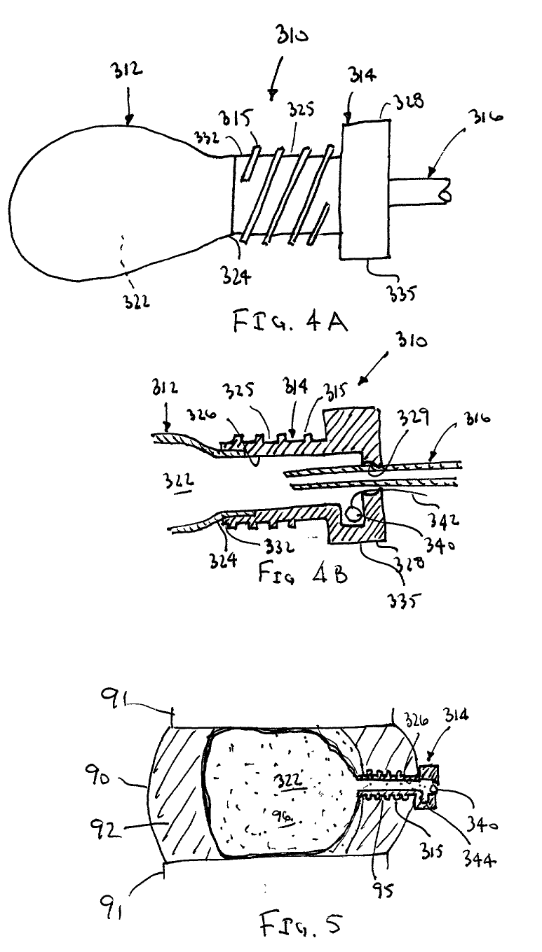

Turning to FIGS. 2A-2I, the apparatus 10 may be used to treat a spinal disc 90, such as that shown in FIG. 2A. The disc 90 is generally disposed between adjacent vertebrae 91, and includes an annulus fibrosis 92 defining an interior region 94 that is substantially filled with nucleus pulposus material. Details of the vertebrae and disc are omitted for clarity, but are well known to those skilled in the art.

First, as shown in FIG. 2B, after gaining access to the disc 90, e.g., using conventional open or minimally invasive surgical methods, an opening 95 is created in the annulus fibrosis 92 to gain access to the interior region 94. For example, a puncture may be created through the annulus fibrosis, a bore may be cut through, or a flap may be created.

As shown in FIG. 2C, at least a portion of the nucleus pulposus may be removed from the interior region 94, thereby defining a cavity 96. This may involve scraping, drilling, coring, or otherwise removing the nucleus pulposus material, e.g., using a scraper, a drill, a screw, a wire or bristle brush, and/or other tool. Alternatively, a fluid or other material may be introduced into the interior region to loosen or otherwise help break up the nucleus pulposus to facilitate its removal. Additional materials and methods may be used to remove nucleus pulposus from within a spinal disc, either alone or in conjunction with one or more of the methods described above, such as those disclosed in U.S. Pat. Nos. 4,439,423 and 4,719,108, issued to Smith, and 3,678,158, issued to Sussman, the disclosures of which are expressly incorporated herein by reference. Preferably, substantially all of the nucleus pulposus is removed from the interior region 94, although, alternatively, only selective portions may be removed. The nucleus pulposus is preferably preserved, e.g., for use in filling the bladder 12, as described further below. Alternatively, the removed nucleus pulposus may be discarded.

As shown in FIGS. 2D and 2E, the apparatus 10 is introduced through the opening 95 into the cavity 96 with the bladder 12 disposed in its collapsed configuration within the sheath 18. The distal end 42 of the sheath 18 is positioned until the bladder 12 is disposed in a predetermined orientation within the cavity 96. This manipulation may be facilitated by external visualization of the marker (not shown) on the apparatus 10, e.g., using fluoroscopy, MRI, and the like. Alternatively, the opening 95 may be sufficiently large that direct visualization may be used. Once properly positioned, the sheath 18 may then be withdrawn, as shown in FIG. 2F, thereby deploying the bladder 12 within the cavity 96.

Fill material may then be introduced into the bladder 12, thereby causing the bladder 12 to expand to its enlarged configuration, as shown in FIG. 2G. Preferably, the fill material includes nucleus pulposus, and more preferably, the fill material includes at least some of the nucleus pulposus material removed from the disc 90. In addition or alternatively, the fill material may include other ingredients, e.g., naturally occurring extra-cellular matrix material, such as intestinal submucosa, stomach submucosa, and bladder submucosa, autologous therapeutics agents, e.g., concentrated growth factors derived from centrifuged plasma obtained from the patient, saline, a pharmaceutical, and/or genetic material. For example, the nucleus pulposus that is removed from the interior region 94 of the annulus fibrosis 92 may be broken down into relatively small particles, e.g., by chopping or other processing, and/or may be mixed with a fluid or other carrier, such as saline, to facilitate its introduction into the bladder 12. Preferably, the fill material is selected to prevent vascularization of the interior region 94, which may otherwise cause nerve growth and, consequently, pain.

Alternatively, the bladder 12 may be filled with a synthetic material, e.g., a polymer, such as sorbathane or other interpenetrating polymer network (IPN) material. Additional information on such materials may be found in “The Development of an Interpenetrating Polymer Network to Contain Mechanically Induced Vibration,” by Maurice Hiles, the disclosure of which is expressly incorporated herein by reference. In a further alternative, IPN material may be delivered directly into the interior region 96 of the disc 90, i.e., without a bladder or other containment, as described further below.

As best seen in FIG. 2G, as the bladder 12 expands, it substantially occupies the cavity 96 from which the nucleus pulposus has been removed. Thus, the bladder 12 may substantially fill any voids within the cavity and/or substantially engage any exposed surfaces, e.g., the exposed surfaces of the vertebrae 91, and/or the inner surface of the annulus fibrosis 92. The bladder 12 may expand and force the vertebrae 91 further apart from one another and/or adjust their relative position, e.g., to remove stress from the annulus fibrosis 92. Thus, the bladder 12 may facilitate treating a disc that is at least partially collapsed or ruptured and/or treating vertebrae that are out of alignment.

Alternatively, the bladder 12 may facilitate healing of an annulus fibrosis, for example, through which fissures and the like have developed. In addition to the nucleus pulposus removed from the interior region 94, any nucleus pulposus that has leaked through such fissures may be removed. In this embodiment, the bladder 12 is preferably substantially nonporous, thereby containing the nucleus pulposus within the bladder 12 while the annulus fibrosis 92 is given opportunity to heal. Preferably, the bladder 12 is bioabsorbable such that the bladder 12 is substantially absorbed by the patient's body after sufficient time for the annulus fibrosis to substantially heal. Thus, once healed, the patient's spinal disc may be restored to a substantially normal, healthy disc.

In a further alternative, a small amount of a flowable fill material (not shown) may be introduced into the cavity 96 before introducing the apparatus 10 and bladder 12 into the cavity 96. For example, a slurry including naturally occurring extra-cellular matrix material, such as intestinal submucosa, stomach submucosa, and/or bladder submucosa, may be introduced into the cavity 96. In addition, or alternatively, the slurry may include a carrier, such as saline, and/or other healing-promoting materials or therapeutic compounds, such as an antibiotic, a steroid, an nsaid, an autologous therapeutics agent, e.g., a concentrated growth factor derived from centrifuged plasma obtained from the patient, and the like.

Thereafter, the bladder 12 may be introduced and filled, as described above. As the bladder 12 is expanded, it may substantially force this external fill material into any gaps, cracks, and/or fissures, e.g., within the annulus fibrosis 92. This may promote healing or remodeling deeper within the annulus fibrosis 92 or other damaged tissue within the disc 90. In addition, the fill material may generate an analgesic effect, as may occur when ECM materials are used, thereby substantially reducing patient discomfort.

Turning to FIG. 2H, once the bladder 12 has been filled to a predetermined pressure, the catheter 16 may be removed. To facilitate disconnecting the neck 24 of the bladder 12 from the distal end 32 of the catheter 16, the pusher member 20 may be advanced distally over the catheter 16 until it abuts or otherwise substantially engages the bladder 12 and/or the neck 24. The catheter 16 may then be withdrawn proximally while the pusher member 20 retains the neck 24 substantially in position, i.e., everted within the interior region of the bladder 12. Once the distal end 32 of the catheter 16 is withdrawn from the neck 24, the sealing member 28 preferably automatically constricts around the neck 24 to substantially seal the opening 26, as shown in FIG. 2H. Alternatively, the neck 24 may be affirmatively closed using a sealing member, such as those described elsewhere herein.

To further facilitate removal of the catheter 16 without pulling the neck 24 from within the bladder 12, the distal end 32 of the catheter 16 may be coated with a lubricious material, such as teflon, and/or the distal end 32 may be tapered to facilitate sliding the distal end 32 out of the neck 24. In a further alternative, the neck 24 and/or opening 26 may be affirmatively sealed, e.g., using an adhesive or other sealant, using RF energy, and the like.

Finally, the pusher member 20 may be withdrawn, and the opening 95 may be closed, thereby substantially sealing the bladder 12 within the annulus fibrosis 92. The opening 95 may be closed by introducing a plug or other closure member (not shown) into the cavity 96 and/or into the opening 95. The plug may be expandable to engage the annulus fibrosis tissue surrounding the opening 95 and/or may otherwise be secured within the opening 95. In addition, or alternatively, an adhesive or other material may be introduced into the opening 95 to substantially seal it. Additional information on closure devices appropriate for closing an opening through an annular fibrosis and methods for using them may be found in application Serial No. ______, filed on the same day as the present application, and entitled “Apparatus and Methods for Closing Openings in Spinal discs” (attorney docket 260/101). The disclosure of this application, and any references cited therein, is expressly incorporated herein by reference.

In an alternative embodiment, for relatively smaller openings, the opening 95 may be closed by applying energy to annular fibrosis tissue surrounding the opening 95. For example, one or more electrodes (not shown) may be provided on the distal end of the catheter 16, as described above. Electrical energy, preferably radio frequency (RF) energy, may be applied to the electrodes, e.g., from an RF generator located outside the patient's body. Thus, as the distal end of the catheter 16 is withdrawn through the opening 95, the electrode(s) may be activated for a predetermined time. This RF energy may contract collagen or other materials in the annulus fibrosis, thereby causing the tissue to close around and substantially seal the opening 95. Additional information on using RF energy to close a passage through tissue may be found in U.S. Pat. No. 5,507,744, 5 issued to Tay et al., the disclosure of which is expressly incorporated herein by reference. Alternatively, other forms of energy may also be used, such as cryogenic energy, microwaves, and the like.

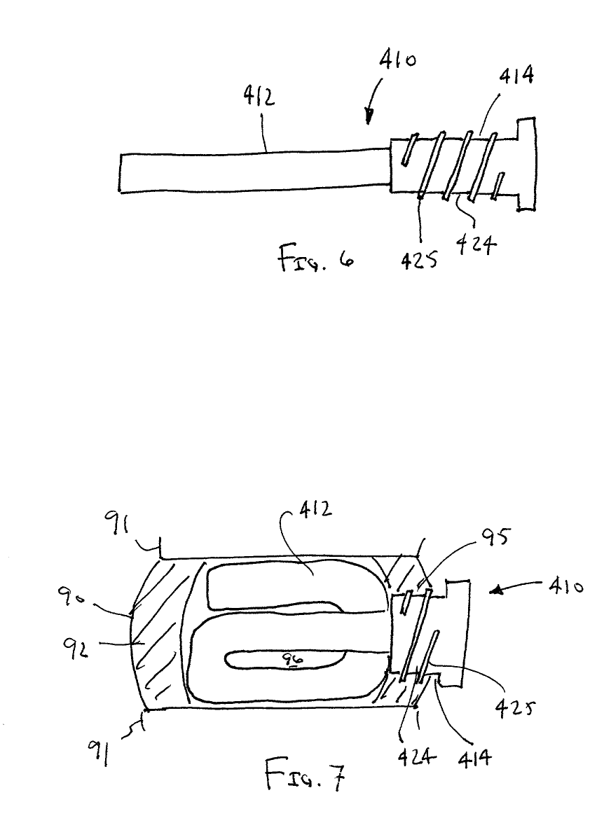

Turning to FIGS. 3A-3D, an alternative method for treating a spinal disc 90 is shown, using an implant 110 that includes a sheet of material 112 and a plug 114, as shown in FIG. 3A. The sheet of material 112 may be formed from a substantially nonporous, bioabsorbable material, defining an outer edge 116, similar to the bladder described above. For example, the sheet 112 may include one or more layers of extra-cellular matrices, such as intestinal submucosa, stomach submucosa, and/or bladder submucosa.

First, similar to the embodiments described above, after gaining access to a disc 90, an opening 95 is created in the annulus fibrosis 92 to gain access to an interior region 94 of the disc 90. At least a portion of the nucleus pulposus may be removed from the interior region 94, thereby defining a cavity 96. The nucleus pulposus may be preserved or may be discarded.

As shown in FIG. 3B, the sheet 112 is introduced through the opening 95 to substantially line the cavity 96. For example, the sheet 112 may be disposed in a collapsed configuration over a rod, catheter, or other elongate member 120. Preferably, an intermediate region of the sheet 112 abuts a distal end 122 of the elongate member 120, and the outer edge 116 of the sheet 112 is disposed proximal to the distal end 122. optionally, a constraint (not shown) may be disposed over the outer edge 116 and/or over other regions of the sheet 112, e.g., to substantially secure the sheet 112 to the elongate member 120.

The distal end 122 of the elongate member 120 may be advanced through the opening 95, thereby introducing the sheet 112 into the cavity 96. The sheet 112 may be disposed in a predetermined orientation within the cavity 96, preferably such that the intermediate region of the sheet 112 is disposed within the cavity 96, while the outer edge 116 of the sheet 112 extends into or through the opening 96. More preferably, the sheet 112 has a size such that the sheet 112 may substantially line the cavity 96, while the outer edge 116 may extend through the opening 96. If a constraint is used, the constraint may be withdrawn to release the sheet 112 from the elongate member 120, whereupon the elongate member 120 may be withdrawn.

As shown in FIG. 3C, the cavity 96 may then be filled with fill material, thereby expanding the sheet 112 to an enlarged configuration, engaging tissue surrounding the cavity 96 and substantially lining the cavity 96. Preferably, the fill material includes nucleus pulposus, and more preferably, the fill material includes at least some of the nucleus pulposus material removed from the disc 90, as described above. In addition or alternatively, the fill material may include other materials as described elsewhere herein, such as autologous therapeutics agents, e.g., concentrated growth factors derived from centrifuged plasma. In a further alternative embodiment, a small amount of a flowable fill material (not shown) may be introduced into the cavity 96 before introducing the sheet 112, similar to the embodiment described above.

If the elongate member 120 is a catheter, the fill material may be introduced through a lumen 122 of the catheter into the cavity 96. Once the bladder 12 has been filled to a predetermined pressure, the catheter 120 may be removed. Alternatively, the elongate member 120 may be removed, and a separate tubular member (not shown) may be advanced through the opening 95 into the cavity 96. Fill material may then be delivered into the cavity 96 through a lumen in the tubular member. Once the cavity 96 has been substantially filled, i.e., to line the cavity 96 with sheet 112, the elongate member 120 or tubular member may be withdrawn.

As shown in FIG. 3D, the plug 114, e.g., an elongate body including a pattern of threads 115 extending along its peripheral surface, may be rotated, and thereby threaded, into the opening 96. Preferably, the body of the plug 114 has a cross-section similar to the cross-section of the opening 96, while the threads 115 have a cross-section substantially larger than the opening 96. Thus, as the plug 114 is rotated, the threads 115 may substantially secure the portion of the sheet 112 extending into the opening 96 against tissue surrounding the opening 96, thereby substantially closing and/or sealing the opening 96. Any excess sheet material may be trimmed and discarded, e.g., either before or after introduction of the plug 114. Alternatively, other plugs or closure devices (not shown) may be delivered into the opening 96 to substantially close and/or seal the opening 96, as described elsewhere herein. In a further alternative, one or more filaments, similar to a purse-string suture, may be attached along the outer edge 116 of the sheet 112, which may be used to draw the outer edge 116 together and substantially seal the fill material within the cavity 96 defined by the sheet 112.

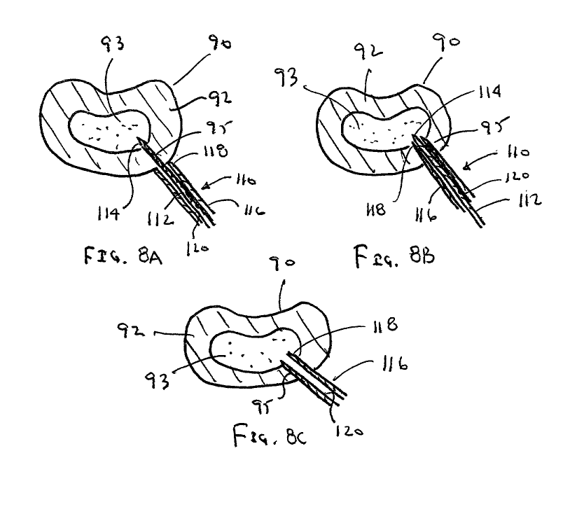

Turning to FIGS. 4A and 4B, yet another embodiment of an apparatus 310 for treating a spinal disc is shown. Generally, the apparatus 310 includes a bladder 312, a plug 314, and a cannula 316. Similar to the embodiments described above, the bladder 312 is expandable from a collapsed configuration to an enlarged configuration, and is preferably formed from a substantially nonporous, bioabsorbable material. The bladder 312 includes a neck 324 communicating with an interior region 322 of the bladder 312.

The plug 314 is a tubular body 325, including a lumen 326 extending between a proximal end 328 and a distal end 332. The neck 324 of the bladder 312 is attached to the distal end 332 of the plug 314, e.g., by an adhesive, sutures, a mechanical fastener, and the like. The plug 314 includes an external thread pattern 315, and may include an enlarged proximal region 335. A sealing element 340 is disposed within the lumen 326 that may selectively open and close the lumen 326. For example, the sealing element 340 may be a ball or other plug that is movable into a pocket 344 within the body 325, e.g., to accommodate insertion of a distal end 320 of the cannula 316 into the lumen 326 and/or to otherwise permit delivery of fill material via the lumen 326 into the bladder 312.

The sealing element 340 may be connected to a filament or wire 342 that extends from the sealing element 340 through the lumen 326 and out the proximal end 328 of the plug member 314. The filament 342 may be used to manually pull the sealing element 340 out of the pocket 344 and into the lumen 326 to close the lumen 326 to fluid flow, as described further below. Alternatively, the sealing element 340 may be connected to a spring element (not shown) that may be connected to a predetermined location of the plug 314. The spring element may be deflected to allow the sealing element 340 to be received in the pocket 344, but may be biased to pull the sealing element 340 into and substantially close the lumen 326.

The proximal end 328 of the body 324 may include a socket 329 for receiving the sealing element 340 therein to substantially close the lumen 326. For example, the socket 329 may have a female mating shape corresponding to the sealing element 340 for positively seating the sealing element 340 in the socket 329 to substantially seal the lumen 326 from fluid flow therethrough.

Turning to FIG. 5, during use of the apparatus 310, an opening 95 may be made in the annulus fibrosis 92 of a spinal disc 90, and nucleus pulposus may be removed to create a cavity 96 within the disc 90, similar to the previously described embodiments. With the bladder 312 in its collapsed configuration, the bladder 312 and the distal end 332 of the plug member 314 may be introduced into the opening 95 until the bladder 312 is disposed within the cavity 96. The sealing element 340 may be pre-loaded within the pocket 344 (not shown in FIG. 5) and/or may be directed into the pocket 344, e.g., during insertion of the distal end 320 of the cannula 316 into the lumen.

The distal end 320 of the cannula 316 may be inserted into the lumen 326 of the plug 314, and fill material, such as the materials described above, may be delivered into the bladder 312 to expand it towards its enlarged configuration and substantially fill the cavity 96. If the sealing element 340 is biased to deploy into the lumen 326 and/or the socket 329, insertion of the cannula 316 into the lumen 326 may direct the sealing element 340 into the pocket 344, thereby opening the lumen 326. Alternatively, the distal end 320 of the cannula 316 may be introduced into the lumen 326 before the bladder 312 and plug 314 are introduced into the disc 90, thereby allowing controlled placement of the sealing element 340 in the pocket 344 and/or placement of the filament 342 in a manner that facilitates access to the filament 342.

Once the bladder 312 is filled to a predetermined pressure, the cannula 316 may be removed, and the sealing element 340 moved into the lumen 326, and preferably into the socket 329. If the sealing element 340 is deployed manually, this may involve pulling the filament 342 until the sealing element 340 is received in the socket 329. Thereafter, any portion of the filament 342 extending from the disc 90 may be trimmed as desired. If the sealing element 342 is connected using a spring element, the sealing element 342 may automatically deploy into the socket 329 upon removal of the cannula 316. Thus, the sealing element 342 may substantially seal the lumen 326, and prevent substantial leakage of fill material from within the bladder 312. In an alternative embodiment, the bladder 312 and plug 314 may be provided separate from one another and deployed independently of one another, similar to the embodiments described above.

Turning to FIG. 6, still another embodiment of an implant 410 is shown for treating a spinal disc that includes an elongate body of fill material 412 and a plug member 414. The body of fill material 412 may be a substantially flexible body formed from material, such as a bioabsorbable material, a material designed to promote regeneration or other healing of the disc, and/or a biocompatible, substantially permanent implant material, similar to the various embodiments described above. For example, the body of fill material 412 may include one or more layers of naturally occurring extra-cellular matrix material and/or nucleus pulposus rolled or packed into a tubular or substantially solid body. The body of fill material 412 may be provided in a predetermined length and/or may be cut to predetermined length. For example, the predetermined length may result in a volume of fill material that substantially matches the volume of an interior of a spinal disc being filled.

The plug member 414 may include an elongate body 424 having a thread pattern 425 extending along the body 424. Alternatively, other external connectors may be provided on the plug member 414 to substantially engage surrounding tissue, such as tines or other tissue engaging elements.

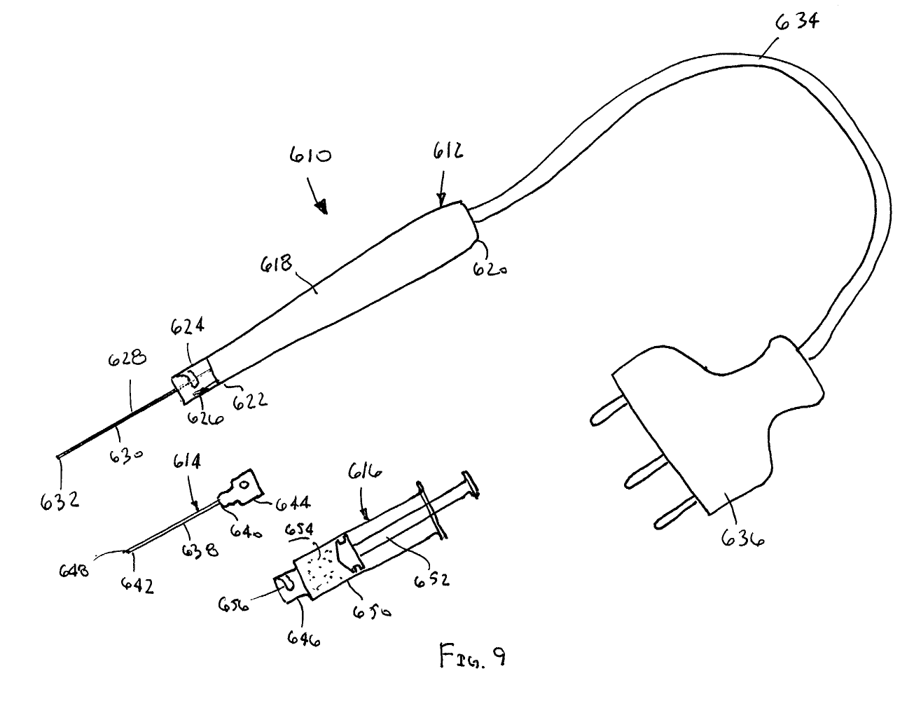

Turning to FIG. 7, the implant 410 is introduced into a spinal disc 90, using a similar method to the embodiments described above. An opening 95 is formed in the annulus fibrosis 92, and at least a portion of the nucleus pulposus is removed to create a cavity 96. The body of fill material 412 is fed through the opening 95 until it substantially fills the cavity 96 and/or the plug 414 is threaded or otherwise engaged within the opening 95. Thus, when the plug member 414 is secured within the opening 95, the body of fill material 412 preferably substantially fills the interior 94 of the disc 90. The implant 410 may be left within a patient's body, e.g., until it eventually is absorbed, e.g., after sufficient time to allow the disc 90 to heal, or substantially permanently.

In a further alternative, an IPN polymer, such as sorbathane, may be implanted directly into an interior of a spinal disc or may even be used to form a prosthetic disc that may replace an entire intervertebral disc. An IPN polymer may allow particular mechanical properties to be selected for the implant, e.g., viscous and/or elastic properties. The viscosity of the polymer may control the level of energy absorption, while the elasticity may dictate the frequency and amplitude at which absorption may occur. An IPN polymer may be customized to optimally set the ratio of these properties to best respond to conditions experienced by an intervertebral disc during normal physical activities. Thus, an IPN may provide substantial advantages over natural rubbers, geometric isomers, and other like materials.

The IPN polymer may be preformed into a body that may be inserted into the interior of the disc, or may be injected or otherwise introduced into the interior of the disc and then cured, e.g., by including a catalyst in the injected material, by exposure to heat, moisture, and the like, as is well known to those skilled in the art. The resulting implant may be a substantially permanent replacement for the nucleus pulposus material within the disc or for the entire disc.

Turning to FIGS. 8A-8C, an apparatus 510 is shown for treating a spinal disc 90 of a patient, e.g., using one or more therapeutic agents. The apparatus 510 generally includes a stylet 512 having a pointed distal tip 514. The stylet 512 is preferably a substantially rigid solid pointed trocar rod or a tubular needle. The stylet 512 may be formed from stainless steel or other material.

The apparatus 510 also includes a tubular sheath 516 having a relatively thin wall that may be slidably disposed over the stylet 512. The sheath 516 preferably has a tapered distal end 518 for facilitating substantially atraumatic advancement of the sheath 516 through tissue. The sheath 516 includes a lumen 520 extending between its proximal end (not shown) and the distal end 518. The sheath 516 may be formed a polymer, such as polyimide.

The proximal end of the sheath 516 may include a seal for substantially preventing backflow of fluids proximally through 5 the lumen 520, but allowing the stylet 512 to be inserted therethrough. In addition, a source of therapeutic agent (not shown) may be connected to the proximal end of the sheath 516, e.g., to a side port (not shown).

As shown in FIG. 8A, the pointed distal tip 514 of the stylet 512 is inserted through the annulus fibrosis 92 to create an opening 95 communicating with the interior region 93. The sheath 516 is advanced over the stylet 512 until the distal end 518 of the sheath 516 is disposed within the interior region 93, as shown in FIG. 8B. As explained above, the distal end 518 of the sheath 516 is preferably tapered to facilitate its advancement over the stylet 512 and through the annulus fibrosis 92.

The stylet 512 is withdrawn from within the disc 90 and the lumen 520, leaving the sheath 516 within the annulus fibrosis 92, as shown in FIG. 8C. One or more therapeutic agents may then be introduced through the lumen 520 of the sheath 516 into the interior region 93. For example, proteoglycans, proteoglycan recruiting materials, materials for inhibiting nerve ingrowth, and the like may be introduced into the interior region 93 of the disc 90, to provide a desired therapeutic effect, to hydrate the nucleus pulposus within the interior region 93, and the like. Alternatively, other compounds, such as any of those described above, may be introduced via the sheath 516.

A single bolos of therapeutic agent may be delivered into the interior region 93, or a series of treatments may be provided. For example, a pump (not shown) may be implanted within the patient's body, that may be connected to the sheath 516. A therapeutic agent may be delivered by the pump into the interior region over a predetermined time, e.g., continuously or in periodic doses.

Upon completion of delivery of the treatment, the sheath 516 may be withdrawn from the interior region 93 and from the disc 90. The opening 95 may then be closed, e.g., by applying energy to annular fibrosis tissue surrounding the passage to close the passage and/or by deploying a closure element, as described above.

Because of the relatively low profile of the sheath 516, the size of the opening 95 used to access the interior region 95 of the disc 90 may be substantially minimized. This may facilitate closing and/or sealing the opening 95 following treatment and minimize the risk of material leaking from the interior region 93, which may cause discomfort or harm to the patient.

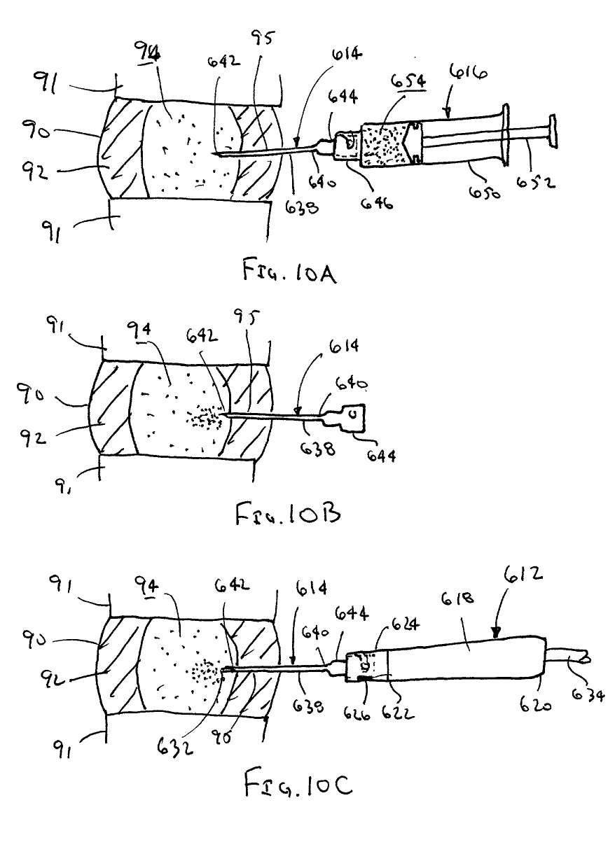

Turning to FIG. 9, an apparatus 610 is shown that may be used to inject a therapeutic agent into an interior region of a spinal disc (not shown). The apparatus 610 may also be used to close a passage through other tissue through which therapeutic agents may be delivered, such as heart tissue, as will be appreciated by those skilled in the art. Generally, the apparatus 610 includes an energy delivery device 612, a needle, 614, a syringe 616, and a source of electrical energy (not shown).

The energy delivery device 612 includes a handle member 618 including proximal and distal ends 620, 622. A connector 624 is provided on the distal end for connecting to a cooperating connector 644 on the needle 614, as described further below. The connector 624 includes an electrically conductive region 626 or is formed entirely from a conductive material for electrically coupling the needle 614 to the source of electrical energy. For example, the connector 624 may be a luer lock, a threaded collar, or other known connector.

An elongate electrode element 628 extends from the distal end 622, preferably substantially coaxially with the connector 624. The electrode element 628 generally includes an electrically insulated outer surface 630 and terminates in an uninsulated distal tip 632. Preferably, the electrode element 628 is a substantially rigid stylet formed from electrically conductive material. The outer surface 630 may be covered with electrically insulating material except for the distal tip 632. Alternatively, the electrode element 628 may be a wire covered with an electrically insulating sleeve or other nonconductive body including an electrode on its distal tip (not shown).

A cable 634 extends from the proximal end 620 of the handle member 618 and terminates with a connector 636 that may be connectable to a source of electrical energy (not shown), preferably a radio frequency (RF) generator. Conductors, such as wires (not shown) may extend through the handle member 618 between the proximal and distal ends 620, 622 for coupling the distal tip 632 of the electrode element 628 and the conductive region 626 of the connector 624 to the source of electrical energy.

The needle 614 may be a conventional hypodermic needle including a tubular body 638 having a lumen (not shown) that extends between proximal and distal ends 640, 642. A luer lock or other connector 644 is provided on the proximal end 640 for connecting to a hub 646 of the syringe 616 and/or for connecting to the handle member 618. The distal end 642 terminates in a pointed tip 648, such as a conventional angled tip that may be used to insert the needle 616 into tissue. The needle 614 is preferably formed from conventional materials, such as stainless steel. Alternatively, the tubular body 638 and all or part of the connector 644 may formed from other electrically conductive materials, as long as the tubular body 638 is electrically coupled to the connector 644.

Preferably, the tubular body 638 and the electrode element 628 have relative lengths such that the distal tip 632 of the electrode element 628 is exposed beyond the distal end 642 of the tubular body 638 when the handle member 618 is connected to the needle 614, as described further below.

The syringe 616 may also be generally conventional, including a barrel 650 and a plunger 652 defining a cavity 654 for containing one or more therapeutic agents. As explained above, the hub 646 includes a complementary luer lock or other connector 656 that may mate with the connector 644 on the needle 614. Alternatively, other containers or sources of therapeutic agents (not shown) may be used that may be connected to the needle 614 to deliver the therapeutic agents into regions beyond or within tissue of a patient.

Turning to FIGS. 10A-10C, the apparatus 610 may be used to inject one or more therapeutic agents through tissue, e.g., into an interior 94 of a spinal disc 90 through the annular fibrosis 92. The therapeutic agent(s) may include drugs or other materials, such as one or more of those described elsewhere herein, including genetic materials, proteoglycans, proteoglycan recruiting materials, materials for inhibiting nerve ingrowth, autologous therapeutic agents, extra-cellular matrix materials, such as intestinal submucosa, stomach submucosa and bladder submucosa, antibiotics, steroids, nsaids, saline, and the like. Other exemplary procedures may include gene-therapy and molecular (drug) treatments using needle injections through tissue, such as for cardiac procedures, e.g., to promote angiogenesis or myogenesis. In a further alternative, the therapeutic agent may be a chemotherapy or other cancer-treatment drug that may be injected into a cancerous region of tissue.

First, as shown in FIG. 10A, the needle 614 is attached to the hub 646 of the syringe 616, and the distal end 642 of the needle 614 is inserted through the annulus fibrosis 92 into the interior region 94 of the disc 90. One or more therapeutic agents are delivered through a lumen (not shown) of the needle 614 into the interior region 94. Once a desired amount of therapeutic agent has been delivered, the syringe 616 may be removed from the proximal end 640 of the needle 614, e.g., by rotating the luer locks, as is know to those skilled in the art. Thus, the needle 614 may remain in the disc 90, as shown in FIG. 10B.

Turning to FIG. 10C, the energy delivery device 612 may then be connected to the needle 614. The elongate element 630 is inserted into the lumen at the proximal end 640 of the needle 614 and advanced therethrough until the distal tip 632 extends beyond the distal end 642 of the needle 614. The connector 624 on the handle member 618 may be secured to the connector 644 on the needle 614, thereby connecting the needle 614 to the handle member 618.

When the connectors 624, 644 are connected, the needle 614 is electrically coupled to the conductive region 626 on the handle member 618. Preferably, because the outer surface of the elongate element 630 is insulated, the needle 614 and the distal tip 632 of the elongate element 630 are electrically isolated to one another, except via tissue surrounding them.

The cable 634 may be connected to a source of energy, such as an RF generator, and electrical energy delivered via the circuit including the distal tip 632, the surrounding tissue, and the needle 614. Thus, a bipolar mode is used to deliver the electrical energy. Alternatively, a monopolar mode may be used, e.g., by placing an electrode pad (not shown) against the patient, e.g., against the patient's skin. The RF generator may be connected to the distal tip 632 and to the electrode pad, thereby delivering electrical energy to the tissue surrounding the distal tip 632.

The electrical energy may be delivered for a predetermined time, e.g., while retaining the needle 614 substantially in place, and upon completion of energy delivery, the needle 614 may be removed from the passage 95. More preferably, the needle 614 is moved along the passage 95 while continuing to deliver electrical energy to the distal tip 632 and the needle 614, thereby closing the passage 95 along a length contacted by the distal tip 632, and preferably substantially along the entire length of the passage 95.

Thus, the energy delivery device 612 may be used to close a passage created using conventional needles and syringes.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

1. An apparatus for treating an interior region of a spinal disc that is accessed through an opening in an annulus fibrosis of the disc, comprising:
an elongate member comprising proximal and distal ends, the distal end having a size for insertion into an opening into a spinal disc;
a sheet of bioabsorbable material on the distal portion of the elongate member such that a peripheral edge of the sheet is disposed generally against the distal portion, the sheet having sufficient surface area for substantially lining an interior region of a spinal disc; and
a closure device for substantially engaging tissue surrounding an opening into a spinal disc, whereby the closure device may substantially seal the opening.

2. The apparatus of claim 1, further comprising a source of fill material for filling an interior region of a spinal disc after the interior region has been lined by the sheet.

3. The apparatus of claim 2, wherein the fill material comprises at least one of nucleus pulposus, an autologous therapeutic agent, and an extra-cellular matrix material.

4. The apparatus of claim 2, wherein the elongate member comprises a tubular member including a lumen communicating between the proximal and distal ends of the tubular member.

5. The apparatus of claim 4, wherein the source of fill material is connectable to the proximal end of the tubular member for delivering fill material through the lumen into an interior region defined by the sheet.

6. The apparatus of claim 1, wherein the closure device comprises a threaded plug.

7. The apparatus of claim 1, further comprising a sheath slidably disposed over the elongate member, thereby disposing the sheet generally against the distal portion of the elongate member.

8. The apparatus of claim 1, wherein the sheet comprises a naturally occurring extra-cellular matrix material.

9. The apparatus of claim 8, wherein the naturally occurring extra-cellular matrix material comprises at least one of intestinal submucosa, stomach submucosa and bladder submucosa.

10. The apparatus of claim 1, wherein the sheet is substantially nonporous.

11. An apparatus for treating a spinal disc, comprising:
a tubular member including a proximal end, a distal end having a size for insertion through an opening into a spinal disc, and a lumen extending between the proximal and distal ends;
a substantially nonporous bladder comprising a neck defining an opening communicating with an interior of the bladder, the neck being detachably connected to the distal end of the tubular member such that the interior of the bladder communicates with the lumen via the opening; and
a source of fill material communicating with the lumen, the fill material comprising nucleus pulposus.

12. The apparatus of claim 11, further comprising a sheath slidably disposed over the tubular member, the sheath comprising a distal region for receiving the bladder therein in a collapsed configuration.

13. The apparatus of claim 11, wherein the bladder further comprises a sealing member for securing the neck over the distal end of the tubular member.

14. The apparatus of claim 13, wherein the sealing member comprises an elastic ring biased to constrict the neck upon withdrawal of the distal end of the tubular member from within the neck.

15. The apparatus of claim 13, wherein the neck is everted within the interior space of the bladder, and wherein the sealing member is disposed around the neck within the interior space.

16. The apparatus of claim 11, further comprising a pusher member slidable along the tubular member, the pusher member configured for directing the neck off of the distal end of the tubular member.

17. The apparatus of claim 16, wherein the pusher member comprises a substantially blunt distal end for engaging the neck when the distal end of the tubular member is withdrawn from within the neck.

18. The apparatus of claim 11, wherein the bladder comprises bioabsorbable material.

19. The apparatus of claim 11, wherein the bladder comprises a naturally occurring extra-cellular matrix material.

20. The apparatus of claim 19, wherein the naturally occurring extra-cellular matrix material comprises at least one of intestinal submucosa, stomach submucosa and bladder submucosa.

21. The apparatus of claim 11, wherein the bladder comprises a substantially inelastic material.

22. The apparatus of claim 11, wherein the bladder is expandable from a collapsed configuration to facilitate introduction into a spinal disc to an enlarged configuration for filling a cavity created within the spinal disc.

23. The apparatus of claim 22, wherein the bladder comprises a disc shape including convex opposing surfaces in the enlarged configuration.

24. The apparatus of claim 11, wherein the distal end of the tubular member comprises an electrode for delivering energy to tissue surrounding a passage through which the tubular member is inserted for closing the passage upon withdrawal of the tubular member.

25. The apparatus of claim 11, wherein the distal end of the tubular member comprises a radiopaque marker.

26. An apparatus for treating a spinal disc, comprising:
an elongate member including a proximal end and a distal end having a size for insertion through an opening into a spinal disc; and
a substantially nonporous, bioabsorbable bladder comprising a neck defining an opening communicating with an interior of the bladder, the neck being detachably connected to the distal end of the tubular member.

27. The apparatus of claim 26, wherein the elongate member comprises a tubular member comprising a lumen extending between the proximal and distal ends, and wherein the interior of the bladder communicates with the lumen via the opening.

28. The apparatus of claim 27, further comprising a source of fill material communicating with the lumen, the fill material comprising at least one of nucleus pulposus, an extra-cellular matrix, an autologous therapeutic agent, an nsaid, a genetic material, a proteoglycan, a proteoglycan recruiting material, a material for inhibiting nerve ingrowth, an antibiotic, a steroid, and saline.

29. The apparatus of claim 26, further comprising a sheath slidably disposed over the elongate member, the sheath comprising a distal region for receiving the bladder therein in a collapsed configuration.

30. The apparatus of claim 26, wherein the bladder further comprises a sealing member for securing the neck over the distal end of the elongate member.

31. The apparatus of claim 30, wherein the sealing member comprises an elastic ring biased to constrict the neck upon withdrawal of the distal end of the elongate member from within the neck.

32. The apparatus of claim 30, wherein the neck is everted within the interior space of the bladder, and wherein the sealing member is disposed around the neck within the interior space.

33. The apparatus of claim 26, further comprising a pusher member slidable along the elongate member, the pusher member configured for directing the neck off of the distal end of the elongate member.

34. The apparatus of claim 33, wherein the pusher member comprises a substantially blunt distal end for engaging the neck when the distal end of the elongate member is withdrawn from within the neck.

35. The apparatus of claim 26, wherein the bladder comprises a naturally occurring extra-cellular matrix material.

36. The apparatus of claim 35, wherein the naturally occurring extra-cellular matrix material comprises at least one of intestinal submucosa, stomach submucosa and bladder submucosa.

37. The apparatus of claim 26, wherein the bladder comprises a substantially inelastic material.

38. The apparatus of claim 26, wherein the bladder is expandable from a collapsed configuration to facilitate introduction into a spinal disc to an enlarged configuration for filling a cavity created within the spinal disc.

39. The apparatus of claim 26, wherein the bladder comprises a disc shape including convex opposing surfaces in the enlarged configuration.

40. The apparatus of claim 26, wherein the distal end of the elongate member comprises an electrode for delivering energy to tissue surrounding a passage through which the elongate member is inserted for closing the passage upon withdrawal of the elongate member.

41. The apparatus of claim 26, wherein the distal end of the elongate member comprises a radiopaque marker.

42. An apparatus for treating a spinal disc, comprising a substantially nonporous, bioabsorbable bladder comprising a neck defining an opening communicating with an interior of the bladder, the bladder being expandable from a collapsed configuration to an enlarged configuration, the bladder having a size and shape to fit within an interior of a spinal disc in the enlarged configuration.

43. The apparatus of claim 42, wherein the bladder further comprises a sealing member for substantially sealing the neck from fluid flow therethrough.

44. The apparatus of claim 43, wherein the sealing member comprises an elastic ring biased to constrict the neck.

45. The apparatus of claim 43, wherein the neck is everted within the interior space of the bladder, and wherein the sealing member is disposed around the neck within the interior space.

46. The apparatus of claim 42, wherein the bladder comprises a naturally occurring extra-cellular matrix material.

47. The apparatus of claim 46, wherein the naturally occurring extra-cellular matrix material comprises at least one of intestinal submucosa, stomach submucosa and bladder submucosa.

48. The apparatus of claim 42, wherein the bladder comprises a substantially inelastic material.

49. The apparatus of claim 42, wherein the bladder comprises a disc shape including convex opposing surfaces in the enlarged configuration.

50. The apparatus of claim 42, wherein the interior of the bladder is filled with material comprising at least one of nucleus pulposus, an extra-cellular matrix, an autologous therapeutic agent, an nsaid, a genetic material, a proteoglycan, a proteoglycan recruiting material, a material for inhibiting nerve ingrowth, an antibiotic, a steroid, and saline.

51. The apparatus of claim 42, further comprising a plug member attached to the neck, the plug member comprising a body having a size for insertion into an opening in a spinal disc, and a connector for engaging tissue surrounding the opening.

52. The apparatus of claim 51, wherein the body comprises a lumen therethrough communicating with the interior of the bladder.

53. The apparatus of claim 51, further comprising a plug element within the body, the plug element deployable for opening and closing the lumen to fluid flow therethrough.

54. A device for closing an opening in a spinal disc, comprising:
an elongate body formed from biocompatible material and comprising a connector on an exterior surface of the body for substantially engaging tissue surrounding the opening, the body comprising a lumen extending between its proximal and distal ends; and
a plug element within the body, the plug element being movable from a pocket within the body into the lumen for substantially sealing the lumen from fluid flow therethrough.

55. The device of claim 54, further comprising a substantially nonporous bladder comprising a neck defining an opening communicating with an interior of the bladder, the neck being attached to the distal end of the body such that the lumen communicates with the interior of the bladder.

56. The device of claim 55, wherein the bladder comprises substantially nonporous material.

57. The device of claim 55, wherein the bladder comprises bioabsorbable material.

58. The device of claim 54, wherein the connector comprises a thread pattern on an exterior of the body.

59. The device of claim 54, wherein the connector comprises a plurality of tissue engaging elements expandable from a contracted condition to an expanded condition.

60. An implant for treating an interior of a spinal disc, comprising:
an elongate body of fill material comprising at least one of nucleus pulposus and naturally occurring extra-cellular matrix material; and
a plug member connected to one end of the elongate body, the plug member comprising a connector on an exterior portion thereof for engaging tissue surrounding an opening through which the body of fill material is introduced for substantially closing the opening.

61. The implant of claim 60, wherein the body of fill material may be cut into a predetermined length corresponding to a volume of an interior of spinal disc within which the body of fill material is introduced.

62. The implant of claim 60, wherein the connector comprises a thread pattern on an exterior of the body.

63. The implant of claim 60, wherein the connector comprises a plurality of tissue engaging elements expandable from a contracted condition to an expanded condition.

64. The implant of claim 60, wherein the fill material further comprises at least one of an autologous therapeutic agent, an nsaid, a genetic material, a proteoglycan, a proteoglycan recruiting material, a material for inhibiting nerve ingrowth, an antibiotic, a steroid, and saline.
